(19) United States
(12) Patent Application Publication
(10) Pub. No.: US 2023/0051289 A1
(43) Pub. Date: Feb. 16, 2023

(54) URGENCY-BASED PATIENT SCHEDULING

(71) Applicant: Dexcom, Inc., San Diego, CA (US)

(72) Inventors: Subrai Girish PAI, San Diego, CA (US); Tomas C. WALKER, Las Vegas, NV (US); Afshan A. KLEINHANZL, San Diego, CA (US); James W. BARMETTLER, Portland, OR (US)

(21) Appl. No.: 17/818,334

(22) Filed: Aug. 8, 2022

Related U.S. Application Data
(60) Provisional application No. 63/231,504, filed on Aug. 10, 2021.

(51) Int. Cl. G16H 40/20

(52) U.S. Cl. CPC G16H 40/20

(57) ABSTRACT

Certain aspects of the present disclosure relate to methods of updating patient scheduling information. In one aspect, the method includes receiving patient data for a patient having a scheduled appointment on a future date, the patient data including a metric value for a biomarker and time and date information associated with the scheduled appointment. The method further includes comparing the metric value with one or more conditions established based at least in part on a patient history of the patient or population health data. The method also includes, after determining that the metric value satisfies at least one of the one or more conditions, rescheduling the scheduled appointment.

### CROSS-REFERENCE TO RELATED APPLICATIONS

This application claims priority to U.S. Provisional Patent Application No. 63/231,504, filed Aug. 10, 2021, which is hereby assigned to the assignee hereof and hereby expressly incorporated by reference herein in its entirety as if fully set forth below and for all applicable purposes.

### BACKGROUND

Management of a patient's health may include visits by the patient to a medical practitioner, for example, for a checkup, in-patient procedure, out-patient procedure, and the like. Such visits, or appointments, may be scheduled days, weeks, or months in advance. In some instances, as the scheduled visit approaches, the state of the patient's health and/or changes therein may render the visit moot or, in other instances, change an urgency of the visit. For example, a diabetic patient can be scheduled for regular checkups during which the medical practitioner may plan to verify whether the patient is maintaining their glucose levels and general health within acceptable (i.e., threshold) levels. In cases where the patient is successfully maintaining their glucose levels and general health within the acceptable levels, however, a scheduled checkup may be unnecessary. Therefore, visiting the medical practitioner in such cases wastes both the patient's and the medical practitioner's time as well as medical resources that could have been provided to other patients in need of a visit.

In certain other cases, after a visit is scheduled, the patient's health may begin to deteriorate significantly, in which case visiting the medical practitioner prior to the scheduled visit may be lifesaving, or at least beneficial to the patient. In yet other cases, a patient may not meet certain requirements for a scheduled visit (such as an in- or out-patient procedure) as the scheduled visit approaches. For example, certain procedures may require that the patient's health (e.g., analyte measurements, or other parameters of the patient's health) be in a certain state (e.g., within a certain range or meet a certain threshold). For example, a diabetic patient is more susceptible to infections from or following a surgery, especially when the patient's measured glucose levels are out of range. Thus, the patient's glucose levels may be indicative of whether the patient's scheduled procedure can be performed as scheduled or should be delayed. When the patient's glucose levels are measured the day of the scheduled procedure, the procedure may be postponed if the glucose levels are not in range, such as elevated or low. However, measuring the patient's glucose levels and canceling the procedure on the day of the scheduled procedure can result in wasted time for the patient and a wasted procedure slot for the clinic as compared to canceling the procedure days in advance of the scheduled.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### SUMMARY

Certain embodiments provide a method for updating patient scheduling information. The method comprises receiving patient data for a patient having a scheduled appointment on a future date, the patient data comprising a metric value for a biomarker and time and date information associated with the scheduled appointment. The method also comprises comparing the metric value with one or more conditions established based at least in part on a patient history of the patient or population health data. The method further comprises, after determining that the metric value satisfies at least one of the one or more conditions, rescheduling the scheduled appointment.

In certain embodiments, the scheduled appointment comprises one or more of a checkup with a medical practitioner, an out-patient procedure, or an in-patient procedure. In certain embodiments, the biomarker comprises one or more of an analyte, a temperature, a health condition, cholesterol measurement, blood pressure, heart rate, or electrical cardiac activity. In certain embodiments, the metric value comprises a value measured by a sensor device or trend data generated by the sensor device over a period.

Certain other embodiments provide another method for updating patient scheduling information. The other method comprises receiving patient data for a patient having a scheduled appointment on a future date, the patient data comprising a metric value for a biomarker as measured by a diagnostic device over a period of time and the scheduled appointment including time and date information. The other method further comprises comparing threshold criteria for the biomarker, the threshold criteria established based at least in part on a patient history of the patient or population health data, to the metric value. The other method also comprises, upon determining that the metric value does not satisfy the threshold criteria, automatically generating a first notification comprising a first indication that the metric value does not satisfy the threshold criteria, a first proposal to reschedule the scheduled appointment to one of earlier or later with respect to the future date, and a request for confirmation of the first proposal.

The other method additionally comprises, upon determining that the metric value does satisfy the threshold criteria, automatically generating a second notification comprising a second indication that the metric value does satisfy the threshold criteria, a second proposal to reschedule the scheduled appointment to the other of earlier or later with respect to the future date, and a request for confirmation of the second proposal. Furthermore, the other method comprises, upon generating the first notification, communicating the first notification to a medical practitioner and, upon generating the second notification, communicating the second notification to the medical practitioner.

In certain embodiments, the scheduled appointment comprises one or more of a checkup with the medical practitioner, an out-patient procedure, or an in-patient procedure. In certain embodiments, the biomarker comprises one or more of a blood glucose measurement, a measured temperature, a health condition, nutrition details, a medication regimen, cholesterol measurement, blood pressure, heart rate, or electrical cardiac activity. In certain embodiments, the metric value comprises a value measured by the diagnostic device or trend data generated based on the metric value over a period. In certain embodiments, the first proposal to reschedule the scheduled appointment comprises one of advancing the scheduled appointment from the future date to a date before the future date or delaying the scheduled appointment from the future date to a date after the future date. In certain embodiments, the second proposal to reschedule the scheduled appointment comprises delaying the scheduled appointment from the future date to a later date or advancing the scheduled appointment from the future date to an earlier date. In certain embodiments, communicating the first notification further comprises communicating the first notification to the patient and communicating the second notification further comprises communicating the second notification to the patient. In certain embodiments, the threshold criteria is further established based on aggregate data from one or more other patient histories with at least one similarity with the patient.

Certain embodiments describe an additional method for updating patient scheduling information. The additional method comprises receiving patient data, measured by a diagnostic device, for a patient having a scheduled appointment on a future date, the patient data comprising a value for a biomarker and receiving threshold criteria for the biomarker, the threshold criteria established based at least in part on a patient history of the patient and corresponding patient data for at least one other patient. The additional method further comprises receiving scheduling information for the patient, the scheduling information including time and date information for the scheduled appointment and, upon receipt of the patient data for the patient having the scheduled appointment, comparing the threshold criteria to a comparison value based on the value for the biomarker relative to the scheduling information for the scheduled appointment and a current date.

The additional method also comprises, upon determining that the comparison value does not satisfy the threshold criteria: automatically generating a first notification to a healthcare provider, the first notification comprising a first indication that that the biomarker does not satisfy the threshold criteria and a first proposal to reschedule the scheduled appointment for a new date and automatically generating a new scheduled appointment for the new date. The additional method additionally comprises upon determining that the comparison value does satisfy the threshold criteria: automatically generating a second alert to the healthcare provider, the second alert comprising a second indication that that the biomarker does satisfy the threshold criteria and a second proposal to reschedule the scheduled appointment for the new date at a later date than the future date and automatically generating the new scheduled appointment for the new date. Furthermore, the additional method comprises, upon generating the first notification, communicating the first notification to the healthcare provider, upon generating the second alert, communicating the second alert to the healthcare provider, and, upon receiving confirmation of the first proposal or the second proposal, communicating details regarding the new scheduled appointment to a healthcare provider.

In certain embodiments, the method further comprises conveying a third alert to the patient that the scheduled appointment has been rescheduled due to a determination that the comparison value does satisfy the threshold criteria. In certain embodiments, the method further comprises conveying a third alert to the patient that the scheduled appointment has been rescheduled due to a determination that the comparison value does not satisfy the threshold criteria. In certain embodiments, the method further comprises generating the comparison value based on: identifying an expected rate of change for the value over a first period and determining the comparison value by applying the expected rate of change for the value to one or more values of the plurality of values for the value in view of the future date. In certain embodiments, the new date is identified based on a prediction of when the biomarker will satisfy the threshold criteria and the current date.

Certain embodiments provide an additional method for proactively notifying at least one of a patient or a medical practitioner. The additional method includes receiving patient data for the patient. The additional method also includes determining that a metric value for a biomarker satisfies one or more conditions. The additional method also includes generating a first notification to the patient and a second notification to a medical practitioner based on the determination. The additional method also includes generating and presenting a set of questions to ask the patient regarding the determination. The additional method further includes receiving and transmitting one or more answers to the set of questions.

Certain embodiments provide an additional method for prepping a patient for a scheduled visit with a medical practitioner. The additional method includes receiving patient data for a patient. The additional method also includes generating a set of questions for the patient to ask a medical practitioner, based at least in part on the patient data. The additional method further includes transmitting the set of questions to a computing device associated with the patient.

### BRIEF DESCRIPTION OF THE DRAWINGS

FIG. 1 illustrates a block diagram of an example health data system, according to certain embodiments disclosed herein.

FIG. 2 illustrates a glucose monitoring system and exemplary mobile devices associated with the example health data system of FIG. 1 in more detail, according to certain embodiments described herein.

FIG. 3 illustrates an example UI that enables a medical practitioner to create and/or modify a rule for use by the example health data system of FIG. 1, according to certain embodiments described herein.

FIG. 4 illustrates a flowchart of operations performed for applying rules to corresponding patient health data, according to certain embodiments described herein.

FIG. 5 illustrates a flowchart of operations performed for monitoring a patient's health and notifying a medical practitioner about the patient's and/or rescheduling a visit that is currently scheduled for the patient, according to certain embodiments described herein.

FIG. 6 illustrates a flowchart of operations performed for monitoring a patient's health and proactively alerting the patient and/or a medical practitioner about a change in the patient's health, according to certain embodiments described herein.

FIG. 7 illustrates a flowchart of operations performed for monitoring a patient's health and proactively prepping a patient for the patient's scheduled visit with a medical practitioner, according to certain embodiments described herein.

FIGS. 8A-8C illustrate an example scenario for prepping a patient for a scheduled visit with a medical practitioner, according to certain embodiments described herein.

FIG. 9 is a diagram of an embodiment of a processing system that performs or embodies certain embodiments described herein.

FIG. 10 is a diagram of an embodiment of a processing system that performs or embodies certain embodiments described herein.

FIG. 11 is a diagram of an embodiment of a processing system that performs or embodies certain embodiments described herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

As described above, the state of a patient's health and changes therein may render a scheduled visit moot or, in other instances, change an urgency of the visit. However, existing patient health data monitoring and scheduling systems are inefficient and technically unable to dynamically monitor the patient's health, automatically determine whether the patient's scheduled visit should be rescheduled, automatically alert and query the patient about the patient's health, automatically prep the patient regarding the patient's scheduled visit, and/or automatically notify a medical practitioner about the patient's health. Such inefficiencies may lead to increased healthcare costs for patients and insurers, lost earnings for medical practitioners, and lost time for patients, among other negatives.

Certain embodiments described herein provide systems, methods, and apparatuses that proactively and dynamically determine whether the patient's scheduled visit can be advanced (moved to a new date closer to a current date), delayed (moved to a new date farther from the current date), or maintained (kept at a scheduled date) as well as whether a medical practitioner should be notified about the patient's health, whether the patient should be alerted regarding a changing condition associated with the patient's health, whether the patient should be queried (or questioned) regarding the changing condition associated with the patient's health, whether to proactively prep the patient for the patient's scheduled visit by providing the patient with a set of questions to ask the medical practitioner during the patient's scheduled visit, etc.

For example, the systems, methods, and apparatuses in certain embodiments enable a clinic (or a similar entity) to remotely and dynamically monitor and analyze the health data (e.g., on a continuous basis) of a patient having an upcoming visit and determine whether the patient's visit can or should be rescheduled or maintained. As discussed, in the example of a diabetic patient, the scheduled visit may be a check-up during which the medical practitioner may plan to verify the patient's glucose levels and schedule a subsequent visit as appropriate. With such a visit, the systems, methods, and apparatuses allow for automatic and pre-emptive monitoring and analysis of the patient's glucose levels over a time period (e.g., a time period prior to the patient's visit, a time period between the patient's last visit and the patient's next scheduled visit, or some other period of time). In an exemplary scenario, the systems, methods, and apparatuses can determine whether the patient's glucose management indicator (GMI) (as an approximation of glycated hemoglobin A1c (HbA1c)) has gone up or down since the patient's last visit or during a prior time period (e.g., last 3 months). When the systems, methods, and apparatuses determine that the patient's glucose levels satisfy certain conditions (e.g., patient's glucose levels are within a predetermined threshold range), the systems, methods, and apparatuses may determine that the patient's visit can be delayed. Delaying the patient's visit in this scenario saves the patient and the medical practitioner time and money by eliminating an unnecessary in-person visit to the medical practitioner. On the other hand, if the system, methods, and apparatuses determine that the patient's glucose levels satisfy certain conditions (e.g., patient's glucose levels are outside a predetermined threshold range), the systems, methods, and apparatuses can maintain or advance the patient's visit. Advancing the patient's visit in this scenario allows the medical practitioner to address a concerning change in the patient's health.

Similarly, where the patient's visit is an in-patient surgical procedure, the systems, methods, and apparatuses in certain embodiments allow for automatically and pre-emptively monitoring and analyzing the patient's glucose levels over the time period (e.g., a time period prior to the patient's visit, a time period between the patient's last visit and the patient's next scheduled visit, etc.). Based on this monitoring and analysis, the systems, methods, and apparatuses may determine whether certain health metrics indicate that the patient will be in appropriate health for the procedure. For example, where the patient's glucose levels are too high during the time period, the systems, methods, and apparatuses can reschedule the in-patient surgical procedure for a later date, thereby saving patient and medical practitioner resources. In another example, automatically and pre-emptively monitoring and analyzing the patient's glucose levels over a time period may show that the patient needs immediate attention or at least an earlier visit to address the patient's condition.

Additionally, the systems, methods, and apparatuses in certain embodiments allow for proactively alerting the patient and/or the medical practitioner to a changing condition associated with the patient's health, based on monitoring and analyzing the patient's glucose levels over the time period. For example, automatically and pre-emptively monitoring and analyzing the patient's glucose levels over the time period may indicate a potential risk with the patient's condition (e.g., recurring hypoglycemic or hypoglycemic metrics indicating the patient is at risk for hospitalization) or worsening control of the patient's condition (e.g., patient's glucose levels has a worsening time in range greater than a threshold). In response to detecting the potential risk with the patient's condition or worsening control of the patient's condition, the systems, methods, and apparatuses can notify (or alert) the patient that a concerning change in the patient's condition has been detected. In addition to notifying the patient, the systems, methods, and apparatuses can automatically query the patient regarding the detected change in the patient's condition. For example, the systems, methods, and apparatuses can generate a set of questions that are designed to gather information regarding the change in the patient's condition to determine a potential cause for the patient's changing condition (e.g., the patient may be asked about the patient's current health, a noticeable trend(s) in the patient's health, etc.). The systems, methods, and apparatuses may collect the patient's responses to the set of questions and share the patient's responses with the medical practitioner, providing the medical practitioner with relevant information to guide discussion with the patient during the patient's scheduled visit. In this manner, the systems, methods, and apparatuses can significantly improve the patient's care, for example, by proactively intervening to notify the patient and/or medical practitioner as to the patient's changing condition and by providing the medical practitioner relevant information (based on user's responses to a dynamically generated set of questions) regarding the patient's changing condition.

Additionally, in certain embodiments, the systems, methods, and apparatuses can proactively prep the patient for the patient's scheduled visit, based in part on monitoring and analyzing the patient's glucose levels over the time period (e.g., a time period prior to the patient's visit, a time period between the patient's last visit and the patient's scheduled visit). For example, the systems, methods, and apparatuses can generate a set of questions for the patient to ask the medical practitioner during the patient's visit. In certain embodiments, the set of questions may be based on trends in the patient's glucose levels over the time period. For example, the systems, methods, and apparatuses may detect that the patient had low blood glucose levels during the time period and may generate a suggested question for the user to ask the medical practitioner related to low blood glucose levels. In this manner, the systems, and methods, and apparatuses can significantly improve the patient's encounter with the medical practitioner during the patient's visit, e.g., by improving the quality of information discussed during the patient's visit, by improving the efficiency (e.g., saving time) of the patient's visit, by providing the patient with questions to guide the dialogue between the patient and the medical practitioner in situations where the patient has forgotten certain questions to ask the medical practitioner and/or may not know which questions to ask the medical practitioner, etc.

Note that although certain examples and embodiments herein are described in relation to a diabetic patient and, therefore, monitoring and analyzing the patient's glucose measurements and related data, the systems, methods, and apparatuses described herein allow for remotely monitoring and analyzing any health metrics (e.g., analyte measurements, heart rate, respiration rate, etc.) for any patient with any condition, determining whether the patient's scheduled visit may need to be changed, determining whether to proactively notify the patient and/or medical practitioner regarding the patient's condition, and determining whether to proactively prep the patient for the patient's scheduled visit.

### Example System

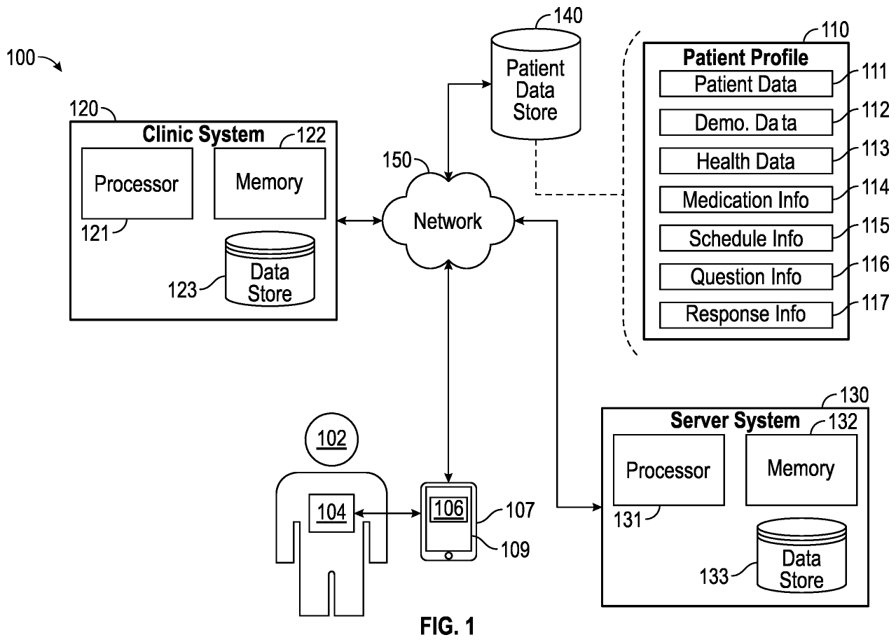

FIG. 1 illustrates a block diagram of an example health data system 100 that enables processing of health data collected, e.g., by a patient computing device (hereinafter referred to as patient device) 107 and accessible by a clinic computing system (hereinafter referred to as clinic system) 120 and/or by a server computing system (hereinafter referred to as server system) 130, according to certain embodiments disclosed herein. A patient 102 may use the patient device 107 to monitor the patient's health and collect patient health data. The health data may be stored in a patient data store 140, for example, as part of a patient profile 110 associated with the patient 102. The patient device 107 is further communicatively coupled to a patient monitoring system 104 that is configured to monitor one or more anatomical parameters of the patient 102, generate data as a result of the monitoring, and transmit the data to the patient device 107. The data generated by the patient monitoring system 104 is similarly monitored and collected as part of the patient health data by the application 106 and is stored in the patient data store 140.

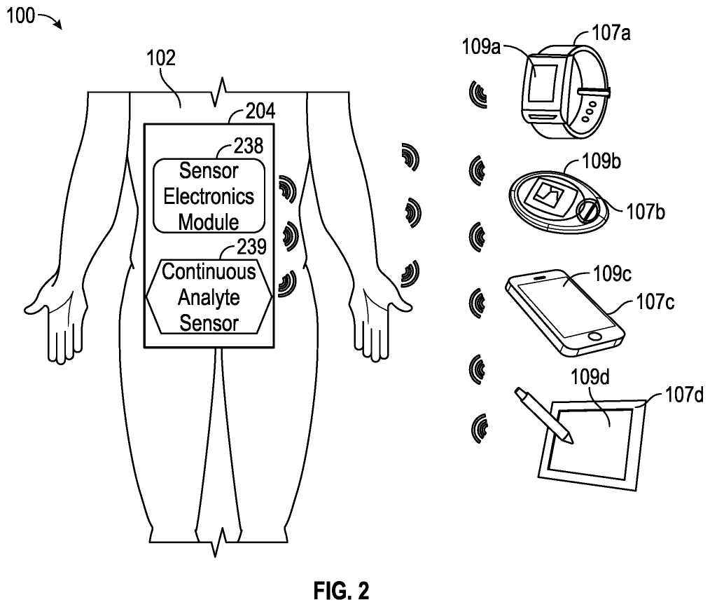

Patient monitoring system 104 may include any wearable or non-wearable devices or systems, such as analyte measurement systems, heart rate sensors, respiration sensors, accelerometers, any other similar type of health monitoring system, or a combination of one or more of these sensors and/or systems. In some examples, the patient monitoring system 104 includes a sensor that is configured to generate measurements of one or more anatomical parameters of the patient 102 on one or more of a continuous, periodic, or on-demand basis. For example, when the patient monitoring system 104 is a glucose monitoring system, the sensor is a glucose sensor configured to measure a concentration of the patient's blood glucose. The patient monitoring system 104 may further include a sensor electronics module that is configured to transmit the measurements to the patient device 107 through a wireless (e.g., Bluetooth) or wired connection. In certain embodiments, the patient monitoring system 104 transmits the measurements to the patient device 107 for use by, for example, the application 106. Further details regarding a glucose monitoring system are provided below with respect to FIG. 2.

In certain embodiments, the application 106 provides a user interface (UI) for display on a display of the patient device 107, via which the patient 102 interacts with one or more of the patient device 107 and/or the patient monitoring system 104. As described above, the application 106 collects health data (e.g., from the patient 102, the patient 102's health care provider, patient monitoring system 104, etc.) and stores the health data in the patient profile 110. The patient profile 110 includes patient data 111 identifying information for the patient 102, such as the patient's name, address, date of birth, and so forth. The patient profile 110 also comprises demographic data 112 for the patient 102, such as one or more of the patient's age, body mass index (BMI), ethnicity, gender, and so forth.

The patient profile 110 additionally includes health data 113 regarding the patient's health, as introduced above, where the health data 113 includes various data points or metrics automatically measured by the patient monitoring system 104, manually entered by the patient 102, provided by clinic system 120 and/or by server system 130, and so forth. In certain embodiments, the health data 113 includes data points from multiple patient monitoring systems 104 or related to various aspects of the patient's health. In certain embodiments, health data 113 includes real-time health metrics, past health metrics, trend data, and/or a rate of change over time developed based on sensor measurements of one or more analytes, other anatomical parameters, etc., over a time period. Examples of health data 113 may include analyte (e.g., glucose, lactate, ketone, potassium, etc.) measurements of the patient collected over time, analyte levels and trends, activity data, food consumption data, metabolic rates, body temperature, heart rate, electrical cardiac activity (e.g., electrocardiogram (ECG)), general health or sickness information, insulin sensitivity, insulin on board, and so forth. In certain embodiments, the health data 113 may further include patient specific thresholds for different anatomical parameters of the patient. For example, the health data 113 may include personalized analyte thresholds (e.g., glucose range, heart rate range, etc.). One or more of these thresholds may be prescribed or set by a medical practitioner for the specific patient, generated by the server system 130 using artificial intelligence (AI)/machine learning (ML) techniques, etc.

The patient profile 110 further includes treatment information 114 such as information associated with a regimen or prescription of any medication taken by the patient 102, details of an exercise regimen, diet information, and the like, for example, to help manage the patient's health. The patient profile 110 further includes schedule information 115 for the patient 102, including details of future visits scheduled for the patient 102, such as a check-up or physical with the medical practitioner, an in-patient procedure, an out-patient procedure, and so forth. The details of the future visits can include details regarding when the visit is scheduled, aspects of the patient's health data related to the visit, and so forth.

The patient profile 110 further includes question information 116, such as a set of questions generated for the patient 102 to ask a medical practitioner during the patient's 102 visit. The question information 116 may include a set of questions provided by the medical practitioner (via clinic system 120), a set of questions provided by the server system 130, or a combination thereof. In certain embodiments, the question information 116 includes a set of questions for the patient 102 to ask the medical practitioner during the patient's visit, a set of questions regarding the patient's condition that the patient 102 should answer prior to the patient's visit, or combinations thereof. The patient profile 110 further includes response information 117, such as a set of responses to one or more of the sets of questions within the question information 116. For example, the response information 117 may include answers to questions generated to gather information regarding a detected change in the patient's condition.

The patient data store 140 stores the patient profiles 110 for a plurality of patients. In certain embodiments, the patient data store 140 corresponds to a storage server that operates in a public or private cloud. In certain embodiments, the patient data store 140 may be included within (or otherwise accessible by) the clinic system 120 and/or the server system 130.

The clinic system 120 is operable at the clinic of the patient's medical practitioner. The clinic system 120 includes a processor 121 configured to execute one or more software applications stored in a memory 122. For example, the processor 121 may execute a scheduling and notification application (hereinafter referred to as “S&N application”). The S&N application may enable the clinic system 120 to dynamically manage schedules of a patient. For example, in certain embodiments, the clinic system 120 may use the S&N application to advance, delay, or maintain a patient's scheduled visit based on information in the patient's profile 110. Similarly, the S&N application may enable the processor 121 to automatically and dynamically generate notifications or messages to medical practitioners, patients, and so forth, based on information in the patient's profile 110.

The clinic system 120 further includes a data store 123 for storing data used by the clinic system 120, such as scheduling and notification rules for use by the S&N application, schedule data for the clinic, patient profile 110, and so forth. The scheduling rules may enable the S&N application to determine whether a patient's scheduled visit should be advanced, delayed, or maintained based on information in the patient's profile 110. In certain embodiments, the scheduling rules may also define for when a visit should be scheduled or rescheduled. The notification rules may enable the S&N application to determine whether to generate a notification regarding a patient. The scheduling rules and the notification rules may be generated by, for example, the medical practitioner. In certain embodiments, the scheduling rules and the notification rules may be patient specific. Further details regarding the S&N application, and corresponding scheduling and notification rules, are described further below.

The server system 130 is operable in a cloud computing environment (e.g., a private or public cloud) and may provide application services and/or features for the patient monitoring system 104 and/or application 106. For example, the server system 130 may receive and store data generated by the patient monitoring system 104 (via application 106), monitor operating parameters of the patient monitoring system 104 (via application 106), push updates to the application 106, etc. The server system 130 includes a processor 131 configured to execute one or more software applications stored in a memory 132. For example, the processor 131 may execute a clinical support application generally configured to monitor and analyze a patient's health data to determine when to proactively alert the patient 102 and/or a medical practitioner regarding a change in the patient's condition, to follow up with the patient 102 regarding the change in the patient's condition, and/or to prep the patient 102 regarding a scheduled visit. In certain embodiments, a patient 102 may opt into receiving services provided by the server system 130 via the application 106. For example, once the patient 102 opts into receiving services provided by the server system 130, the server system 130 may be able to access information in the patient's profile 110, information stored in clinic system 120 (e.g., data store 123), etc.

The clinical support application may enable the server system 130 to proactively alert the patient 102 and/or the medical practitioner regarding a change in the patient's condition, based on monitoring and analysis of the patient's health data (e.g., health data 113) during a time period prior to the patient's scheduled visit. The clinical support application may use a set of rules to determine when the patient's health data satisfies a condition for alerting the patient 102 and/or the medical practitioner. The set of rules may include one or more predetermined rules provided by the medical practitioner (via the client system 120), one or more rules generated using artificial intelligence/machine learning (AI/ML) techniques (via the server system 130), or combinations thereof.

In certain embodiments, the clinical support application also enables the server system 130 to generate a set of questions to ask the patient 102 regarding the detected change in the patient's condition. Such a set of questions may include one or more predefined questions from a standard questionnaire (e.g., standard questionnaire forms, such as Diabetes Treatment Satisfaction questionnaire, CGM (continuous glucose monitor) Usability questionnaire, SPOTLIGHT questionnaire, etc.), one or more predefined questions provided by the medical practitioner, one or more questions generated using AI/ML techniques, or combinations thereof.

Additionally or alternatively, the clinical support application also enables the server system 130 to proactively prep a patient 102 for a scheduled visit by generating a set of questions for the patient 102 to ask the medical practitioner during the patient's scheduled visit. Such a set of questions may include questions selected from a set of predefined set of questions based on analysis of the patient's health data in the time period prior to the patient's scheduled visit. For example, assuming the patient 102 has low glucose levels in the time period prior to the patient's scheduled visit, the clinical support application may select, from a predefined list of questions, questions pertaining to glucose levels for the patient 102 to ask the medical practitioner during the patient's scheduled visit.

The server system 130 further includes a data store 133 for storing data used by the server system 130, such as predefined rules and/or AI/ML models used by the clinical support application, schedule data for the clinic, patient profile 110, and so forth. Note the techniques performed by the server system 130 for proactively alerting the patient 102 and/or the medical practitioner, generating questions to ask the patient 102 regarding a change in the patient's condition, generating questions to prep the patient 102 for the patient's scheduled visit, etc. are described further below.

The clinic system 120, the patient device 107, the patient data store 140, and the server system 130 are communicatively coupled via a network 150. The network 150 may be a local area network (LAN), an intranet, a wide area network (WAN), the Internet, or any other group of connected computing devices, such as the patient device 107 and the clinic system 120. In certain embodiments, though not shown, the network 150 communicatively couples additional patient devices 107 for the same or other patients and/or couples additional clinic computing systems 120 for the same or different clinics in the system 100.

FIG. 2 illustrates an example of patient monitoring system 104 and exemplary mobile devices 107a-107d of the example health data system 100 of FIG. 1 in more detail, according to certain embodiments described herein. Note that the patient device 107 of FIG. 1 may be any one of mobile devices 107a-107d. In other words, any one of mobile devices 107a-107d may be configured to execute an application, such as the application 106. In the example of FIG. 2, the patient monitoring system 104 is a glucose monitoring system that is communicatively coupled to one or more of the mobile devices 107a-107d. However, the patient monitoring system 104 may instead or additionally include any other type of physiological monitoring systems that may be used to monitor and/or treat a patient. Example physiological monitoring systems include electrical or optical heart rate monitoring systems, pulse oximeters, temperature monitors, blood pressure monitors, hydration monitors, various analyte sensors (e.g., lactate sensor, ketone sensor, glucose sensor, cholesterol sensor, etc.), and so on, configured to measure a biomarker (also referred to a biometric marker) or anatomical parameter, such as blood glucose, a temperature, a health condition, nutrition details, a medication regimen, cholesterol, blood pressure, heart rate, and electrical cardiac activity (e.g., ECG) among other things.

By way of an overview and an example, assuming the patient monitoring system 104 is ae glucose monitoring system 204, the glucose monitoring system 204 may be implemented as a microcontroller that performs sensor measurements, generates analyte data (e.g., by calculating values for continuous glucose monitoring data), and engages in wireless communications (e.g., via Bluetooth and/or other wireless protocols) to send such data to remote devices, such as one or more of the mobile devices 107a-107d. Various glucose sensors, such as an on-skin sensor assembly or an implanted sensor assembly, in certain embodiments, is used in connection with glucose monitoring system 204.

In certain embodiments, the glucose monitoring system 204 includes a sensor electronics module 238 and a glucose sensor 239 associated with the sensor electronics module 238. In certain embodiments, the sensor electronics module 238 includes electronic circuitry associated with measuring and processing sensor data or information generated by the glucose sensor 239, including algorithms associated with processing and/or calibration of the sensor data/information. The sensor electronics module 238 is electrically and mechanically connected to the glucose sensor 239 and can be integrated with (i.e., non-releasably attached to) or releasably attachable to the glucose sensor 239.

The sensor electronics module 238 includes hardware, firmware, and/or software that enable measurement and/or estimation of levels of glucose in the patient, via the glucose sensor 239. For example, the sensor electronics module 238 can include a power source for providing power to the glucose sensor 239, other components useful for signal processing and data storage, and a telemetry module for transmitting data from the sensor electronics module 238 to one or more display devices. Electronics can be affixed to a printed circuit board (PCB) within the glucose monitoring system 204, or platform or the like, and can take a variety of forms. For example, the electronics can take the form of an integrated circuit (IC), such as an Application-Specific Integrated Circuit (ASIC), a microcontroller, a processor, and/or a state machine.

The sensor electronics module 238 may include sensor electronics that are configured to process sensor information, such as sensor data, and generate transformed sensor data and displayable sensor information. The glucose sensor 239 is configured to measure a concentration or level of glucose in the patient 102. In certain embodiments, the glucose sensor 239 comprises a continuous glucose sensor, such as a subcutaneous, transdermal (e.g., transcutaneous), or intravascular device. The glucose sensor 239 can use any method of glucose-measurement, including enzymatic, chemical, physical, electrochemical, spectrophotometric, polarimetric, calorimetric, iontophoretic, radiometric, immunochemical, and the like.

With further reference to FIG. 2, one or more of the mobile devices 107a-107d can be configured for displaying (and/or alarming) displayable sensor information that may be transmitted by the sensor electronics module 238 (e.g., in a customized data package that is transmitted to the display devices based on their respective preferences). The mobile devices 107a-107d may respectively include a display, such as touchscreen display 109a-109d, for displaying a graphical UI of the application 106, which may present sensor information and/or data to the patient 102, receive inputs from the patient 102, and/or update details of a patient profile 110. In certain embodiments, the graphical UI of the application 106 may present questions for the patient 102 to answer regarding a change in the patient's condition, receive answers from the patient 102 to the presented questions, present questions for the user to ask a medical practitioner during a scheduled visit, etc. In certain embodiments, the mobile devices include other types of user interfaces such as an audio interface instead of or in addition to a touchscreen display for communicating sensor information to the patient 102 of the mobile device and/or receiving user inputs. In certain embodiments, one, some, or all of mobile devices 107a-107d are configured to display or otherwise communicate the sensor information as it is communicated from the sensor electronics module 238 (e.g., in a data package that is transmitted to respective display devices), without any additional prospective processing required for calibration and/or real-time display of the sensor data.

One or more of the mobile devices 107a-107d depicted in FIG. 2 may include a custom or proprietary display device, for example, an analyte display device 107b, specifically designed for displaying certain types of displayable sensor information associated with the data received from the sensor electronics module 238 (e.g., a numerical value and/or an arrow, in certain embodiments). In certain embodiments, one or more of the mobile devices 107a-107d includes a smartphone, such as mobile phone 107c, based on an Android, iOS, or another operating system configured to display a graphical representation of the continuous sensor data (e.g., including current and/or historic data).

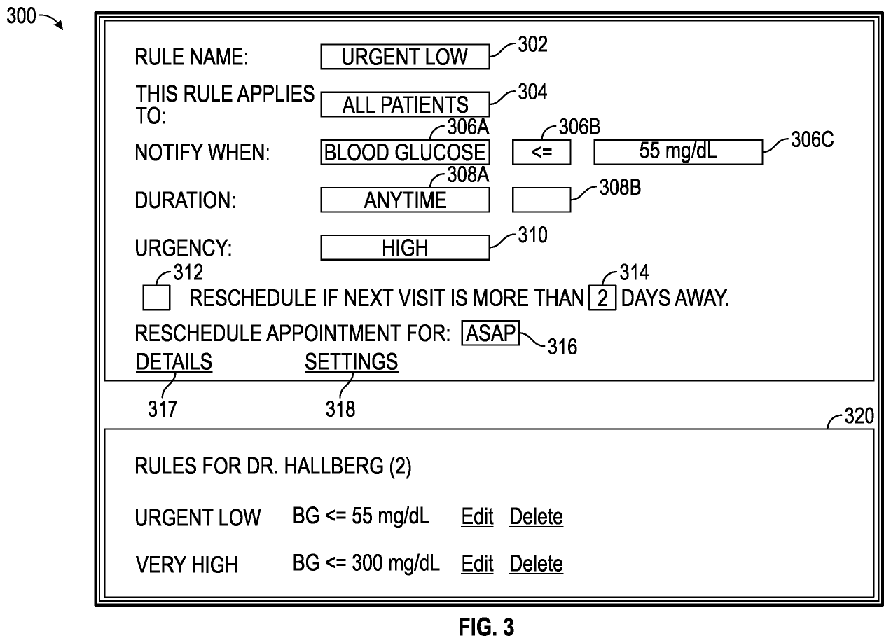

As introduced above, the clinic system 120 employs notification rules to determine whether to generate a notification regarding a patient and scheduling rules to determine whether a patient's scheduled visit can be advanced, delayed, or maintained. An example of a UI used to generate and edit such rules is provided with respect to FIG. 3.

FIG. 3 illustrates an example UI 300 that enables a medical practitioner to generate and/or modify a rule (for example, a notification and/or a scheduling rule) to apply to information stored in a patient profile 110, such as health data 113 and schedule info 115, for a patient. In certain embodiments, the UI 300 may be provided by the S&N application for display on a display device of the clinic system 120. The UI 330 may provide a plurality of fields that allow for creating/modifying rules, identifying to which one or more patient each rule applies, and defining one or more of a condition, a corresponding action to take if/when the condition occurs, and associated details for each rule.

The example UI 300 of FIG. 3 allows a user to create a rule that is both a notification rule as well as a scheduling rule. As discussed above, a notification rule created through UI 300 can be used by clinic system 120 to determine when to notify the medical practitioner about a patient. For example, the clinic system 120 may apply conditions (e.g., as indicated by fields 306A-306C) created by the user to information in the patient's profile to ascertain whether/when the medical practitioner should be notified about the patient's condition. On the other hand, a scheduling rule created through UI 300 can be used by clinic system 120 to determine whether/when to reschedule a patient's visit. For example, the clinic system 120 may apply conditions (e.g., as indicated by fields 306A-306C and 312-316) created by the user to information in the patient's profile to ascertain whether or not to reschedule a patient's visit. The rule created through UI 300 is both a notification rule as well as a scheduling rule because it defines conditions for whether/when a notification as well as rescheduling needs to occur.

As shown, the UI 300 includes a field 302 for a name associated with the rule being created or modified via the UI 300. The medical practitioner may use the field 302 to input any alphanumeric name to uniquely identify the rule. At field 304, the medical practitioner may identify a group of one or more patients (e.g., having patient profiles 110 stored in the patient data store 140) to which the rule applies. In some instances, the medical practitioner identifies all clinic patients (as shown in FIG. 3), only patients for the medical practitioner, a specific patient of the clinic, patients having visits scheduled within an upcoming period, and so forth. In certain embodiments, the field 304 comprises a dropdown menu with multiple available selections. For scheduling rules, the field 304 may enable identification of patients having scheduled visits within specified periods. As shown in the example UI 300 of FIG. 3, the rule is named “Urgent Low” at field 302 and applies to “All patients” at field 304.

The UI 300 further comprises fields 306A-C with which the medical practitioner identifies certain conditions for the rule. More specifically, the fields 306 enable the medical practitioner to establish conditions for when the named rule in field 302 will apply to the identified patient(s) of the field 304. For example, the conditions define the circumstances under which (1) a notification to the medical practitioner is to be generated for the patient by the processor 121 and (2) the patient's scheduled visit should be rescheduled.

The field 306A enables the medical practitioner to identify a biomarker, such as “blood glucose,” “estimated HbA1c (eA1c)”, “weight,” “insulin,” and the like. The field 306B further enables the medical practitioner to define a comparator, such as “less than or equal to,” “greater than or equal to,” “less than,” “greater than,” “equal to,” and so forth, and a biomarker threshold value. The field 306C provides a dropdown menu with multiple available selections for each field. In certain embodiments, the available selections for the comparator and/or threshold values of the fields 306 may be dependent on the selected biomarker, where different biomarkers have different available selections for the comparator and the biomarker threshold value. In certain embodiments, the biomarker threshold is established based on the patient's specific health circumstances and history as indicated by the patient's profile 110 (e.g., patient's health data 113, etc.) or based on population health data associated with similar patients. For example, the blood glucose biomarker threshold for a diabetic patient that is 55 years old may be generated based on average blood glucose levels for a number of other diabetic patients between 50 and 60 years of age. As shown in the example UI 300 of FIG. 3, the rule identifies the biomarker of “Blood glucose” at field 306A with the comparator “<=” in field 306B and a corresponding value of “55 mg/dL” at field 306C.

Field 308A enables the medical practitioner to establish a duration of time over which the rule will apply. For example, the medical practitioner can establish the duration as being “Unlimited” or “Anytime,” meaning the rule will apply until turned off, “[f]or at least . . . ,” meaning the rule will apply for an identified time, and so forth. Field 308B enables the medical practitioner to define the duration of time for a selection other than “Unlimited” or “Anytime” at field 308A. The fields 308A and 308B may comprise dropdown menus from which selections are made by the medical practitioner. As shown in the example UI 300 of FIG. 3, the rule identifies the duration as “Anytime” at field 308A with no duration identified at field 308B.

The UI 300 may further provide field 310, which enables the selection of an urgency of the defined rule, as selected by the medical practitioner from a dropdown menu. The urgency may identify one or more of an urgency indicator of any notification generated based on the rule or how quickly the notification is generated and conveyed to the medical practitioner. Urgency selections may include “very high,” “high,” “medium,” “low,” “very low,” and so forth. As shown in the example UI 300 of FIG. 3, the rule identifies the urgency as “High” at field 310.

The UI 300 further provides fields for defining conditions for rescheduling a patient visit, if appropriate. For example, fields 312-316 can further define when to reschedule an existing patient visit if the health-related conditions, as defined by fields 306A-306C, are met. For example, the medical practitioner uses the field 312 (e.g., a selector box) to enable rescheduling of the existing patient visit. When the field 312 enables rescheduling, the medical practitioner uses field 314 to identify what visits to reschedule (e.g., scheduled visits within a certain period, such as two days) and field 316 to identify a window of time during which the visit can be rescheduled (e.g., “ASAP,” “next week,” “next month,” and so forth. In the example shown in FIG. 3, selection of the field 312 defines conditions for rescheduling the patient's visit. If field 312 is not selected the rule created though UI 300 would be a notification-only rule. However, selecting field 312 converts the rule into a notification and scheduling rule. Field 314 indicates that if the patient's visit is scheduled more than 2 days away, the patient visit needs to be rescheduled “ASAP,” as selected by the medical practitioner using field 316. In other words, FIG. 3 shows an example of a rule that advances a patient's visit if the patient's condition is deteriorating and the patient is not already scheduled to visit the medical practitioner in the next 2 days.

The UI 300 may further comprise details selector 317 and settings selector 318. The details selector 317 may enable the medical practitioner to select details for the rule, such as details of the urgency selection of field 310. Examples of such details include ways in which notifications may be provided to a medical practitioner (e.g., at an identified phone number, email notifications at one or more email addresses, names and contact information of medical practitioners to be contacted based on the notification rule, and so forth). The settings selector 318 enables selection of various UI 300 settings, such as display settings, units for display, and the like.

In certain embodiments, the UI 300 may include a screen or portion 320 indicating existing rules for a particular medical practitioner or clinic. The screen 320 may indicate a summary of aspects of the existing rules and options to edit aspects of or delete each existing rule. As introduced above, the selected biomarker determines available selections for the comparator and/or biomarker threshold values of the fields 306. Furthermore, the available range or value for of the selected biomarker may vary based on an amount of time until the scheduled visit. For example, when the patient's visit is a month away, more threshold biomarker values may be available for selection as compared to when the patient's visit is a week away. While the UI 300 depicts dropdown menus and selection boxes, the UI 300 may utilize various modes of user data input and selection.

Note that FIG. 3 illustrates only one example of a UI 300 for creating a rule. In other examples, a UI may be provided for creating only a scheduling rule. In yet other examples, a UI may be provided for creating only a notification rule. In yet other examples, the server system 130 may provide (or generate) a notification rule and/or scheduling rule for the patient 102. In such examples, the server system 130 may automatically populate the fields 306A-306C and 312-316 using one or more AI/ML techniques to create the notification rule and/or scheduling rule for the patient 102. Also, FIG. 3 illustrates only one example of a rule (a notification and scheduling rule in this case). A wide variety of rules may be created for automatically monitoring (e.g., on a continuous, periodic, etc., basis) one or more patients' health/condition and determine when/whether to notify a medical practitioner and/or a patient 102 and/or reschedule the one or more patients' visits.

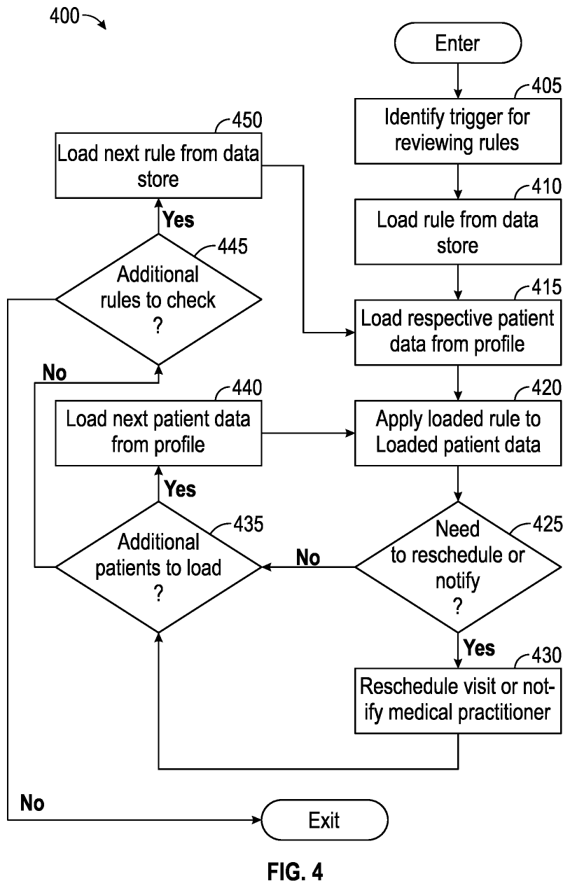

The clinic system 120 may use the S&N application to monitor a patient's health and condition by examining (e.g., continuously, periodically, on-demand, etc.) information that is provided in the patient's profile 110 and apply corresponding rules that have been defined for the patient for the purpose of notifying a corresponding medical practitioner and/or scheduling or rescheduling any patient visits, as dictated by the rules. The clinic system 120 can apply the rules periodically, continuously, or on-demand to the patient profile 110. For example, the clinic system 120 may review health data 113 and schedule info 115 in patient profiles 110 of patients having scheduled visits during a defined future period to determine whether the scheduled visits should be maintained or rescheduled. Based on details of the schedule info 115 and the health data 113, the clinic system 120 may identify those patients whose scheduled visits are to be rescheduled. Further details are provided below with respect to FIG. 4 below.

FIG. 4 illustrates a flowchart of operations 400 performed for applying rules to patient data, according to embodiments described herein. For example, operations 400 correspond to at least part of an algorithm applied by the clinic system 120 using the S&N application introduced above. Note that although blocks of operations 400 are described herein as being performed by the clinic system 120 of FIG. 1, operations 400 may similarly be performed by any other system or clinic system with different components.

At block 405, the clinic system 120 identifies a trigger for automatically applying one or more rules. The trigger may be generated based on one or more of new health data being stored in one or more patient profiles in data store 140, a scheduled event, a request from a medical practitioner, and the like. For example, a scheduled event may comprise an automatic daily review to identify patients having patient visits scheduled for the upcoming week. This daily review may automatically trigger application of the one or more rules to patient health data 113 for the identified patients having a scheduled visit in the next week. In this example, each day, patients having visits scheduled, e.g., in the next seven days are automatically identified and the one or more rules are automatically applied to the identified patients.

At block 410, the clinic system 120 loads the identified rule from, for example, the data store 123. Where the rules are applied one at a time, the rules may be loaded individually. Where multiple rules are applied at once in parallel (not shown), multiple rules may be loaded in parallel. For example, a rule having a biomarker of temperature and a rule having a biomarker of blood glucose (e.g., in fields 306A of FIG. 3) may be applied in parallel to the same identified group of patients (e.g., in field 304 of FIG. 3) because the patient's temperature and blood glucose may both be considerations in rescheduling a patient visit. Thus, where the rules both apply to the same patients, the parallel application may save from loading the same patient data multiple times. The rule may be loaded into the memory 122 for application by the processor 121 to patient data, as described further below.

At block 415, the clinic system 120 loads respective health data (e.g., health data 113) for a current patient to whom the loaded rule applies into the memory 122 from, for example, the current patient's profile 110. For example, the clinic system 120 may determine that the loaded rule applies to the current patient based on determining that patient is part of an identified group of patients. Where the rule indicates “All patients,” then the clinic system 120 may, one by one, load health data for each patient of the clinic. Where the rule identifies “Diabetic patients,” then the clinic system 120 may, one by one, load health data for each of the clinic's diabetic patients as identified in the patients' profiles 110. In certain embodiments, the loaded health data comprises data points, trends, and the like related to the biomarker of one or more conditions of the loaded rule. For example, where the biomarker identified by the rule is blood glucose, the loaded health data for a patient can comprise at least one or more of the patient's most recent blood glucose level(s) or trend data indicating a recent trend of the patient's most recent blood glucose levels.

At block 420, the clinic system 120 applies the loaded rule to the patient data loaded into the memory 122. In certain embodiments, applying the loaded rule to the loaded patient data involves comparing a metric value from the loaded patient health for a biomarker identified by the rule (e.g., in field 306A of FIG. 3) to the defined comparator and the corresponding value (e.g., from fields 306B and 306C of FIG. 3) from the loaded rule. For example, the UI 300 of FIG. 3 indicates a rule having a biomarker of “Blood glucose” and a comparator and corresponding value of “greater than or equal” to “55 mg/dL. Applying this rule as the loaded rule to the loaded patient data comprises comparing the patient's blood glucose level (for example, a most recent blood glucose level or an average of the last certain number of blood glucose levels) to 55 mg/dL using comparator “greater than or equal.” In certain embodiments, comparing the metric value comprises comparing a single data point or a trend generated based on a plurality of data points related to the biomarker.

At block 425, the clinic system 120 determines whether to reschedule a visit for the current patient or notify a medical practitioner based on the application of the rule at block 420. If the loaded rule is a notification rule, then the clinic system 120 determines whether to notify the medical practitioner based on block 420. If the loaded rule is a scheduling rule, then the clinic system 120 determines whether to reschedule the visit based on block 420. If the loaded rule is both a notification and scheduling rule, then the clinic system 120 determines whether to notify the medical practitioner and reschedule the visit based on block 420.

As an example, where the rule identifies “Blood glucose” as the biomarker and a comparator and corresponding value are “greater than” and “200 mg/dL,” the clinic system 120 determines whether, e.g., a most recent blood glucose level or average of the recent glucose data measurements from loaded health data for a patient exceeds the 200 mg/dL biomarker threshold value. When the patient's blood glucose level is greater than 200 mg/dL and the patient's scheduled visit is a surgical procedure, a high blood glucose level during surgery may result in higher risk for infection, and the clinic system 120 may reschedule the surgical procedure to a later date to give the patient's blood glucose level time to reduce to an acceptable value.

In another example, where the rule identifies “Blood glucose” as the biomarker and a comparators and corresponding values are “less than 150 mg/dL and larger than 90 mg/dL” the clinic system 120 determines whether a most recent blood glucose level or trend data from loaded health data for a patient falls in that range. If yes, a blood glucose level in that range may indicate that the patient's blood glucose level is in an expected or healthy state. Therefore, if the patient's visit is just a regular visit for the medical practitioner to address the patient's glucose levels, the clinic system 120 may reschedule the visit to a later date given the patient's blood glucose level is in a generally expected range.

In yet another example, where the rule identifies “Blood glucose” as the biomarker and a comparator and corresponding value of “less than” and “55 mg/dL,” the clinic system 120 determines whether, e.g., a most recent blood glucose level or average of the recent glucose data measurements from loaded health data for a patient is less than the 55 mg/dL biomarker threshold value. As a blood glucose level less than 55 mg/dL may indicate that the patient's blood glucose level is at a concerning level, the clinic system 120 may reschedule the patient's visit to an earlier date given the concern. In certain examples, the patient in this situation may not even have any scheduled appointments. In such examples, when the clinic system 120 determines that the patient's blood glucose level is concerning, the clinic system 120 may (e.g., automatically) schedule the first available appointment for the patient.

At block 430, the clinic system 120 reschedules the patient's visit and/or notifies a medical practitioner. For example, rescheduling the patient's visit may comprise the clinic system 120 identifying, from the schedule data for the clinic in the data store 123, a next available date and time that is appropriate for the patient's visit. As an example, if the rule defines that the visit should be rescheduled for more than two weeks in the future, then the clinic system 120 executing the S&N application may identify a next available date and time from the schedule data that is more than two weeks in the future.

In certain embodiments, the clinic system 120 may automatically reschedule a visit and, in certain other embodiments, the clinic system 120 may reschedule a visit in response to receiving confirmation from a medical practitioner. For example, after having determined to reschedule a visit, the clinic system 120 may generate a message to a medical practitioner including information regarding the proposed rescheduling and requesting that the medical practitioner confirm or approve the rescheduled visit. In certain embodiments, the message may include a visual indication of the current scheduled visit, for example, on a calendar, with details of the visit, such as what the visit is for, associated biomarkers, and so forth. The message may further include a visual indication of the proposed rescheduled visit, such as on a calendar, with details as to why the visit is being rescheduled, including results of the comparison performed at block 420. In certain embodiments, the visual indication may show that the current scheduled visit is proposed to be replaced by or rescheduled to the proposed rescheduled visit.

Once the message is sent to the medical practitioner, the clinic system 120 may receive confirmation from the medical practitioner to reschedule the visit, in response to which the clinic system 120 may reschedule the visit and update the schedule data in the data store 123 and the schedule information 115 in the patient's profile 110 with the rescheduled visit. In certain embodiments, upon updating the schedule data in the data store 123 and the schedule information 115 in the patient profile 110, the clinic system may generate a message to the patient indicating that the patient's visit has been rescheduled. Examples of conditions that result in rescheduling of the patient's visit are provided below for circumstances for advancing a visit or delaying a visit.

In certain embodiments, the clinic system 120 executing the S&N application may generate a notification to the medical practitioner based on, for example, the details 317 of the loaded rule.

At block 435, the clinic system 120 determines whether the loaded rule is applicable to any additional patients. For example, where the loaded rule has been applied to the loaded health data for the current patient, the clinic system 120 determines whether the loaded rule is to be applied to any additional patients. If the loaded rule is applicable to additional patients, then the operation 400 proceeds to block 435. If not, then the operation 400 proceeds to block 440.

At block 440, the clinic system 120 loads the next health data for a next patient to which the loaded rule applies into the memory 122 from the corresponding patient profile 110. Blocks 420 and 425 are repeated for the next patient and the next patient's loaded health data. Blocks 420-435 are repeated for all subsequent patients to which the loaded rule applies until the loaded rule is applied to all relevant patients, at which point the operation 400 proceeds to block 440.

At block 445, the clinic system 120 determines whether there are additional rules to load into the memory 122 and apply against patient health data. If there are additional rules to load, the operation 400 proceeds to block 445. If there are no additional rules to load, the operation 400 ends.

At block 450, the clinic system 120 loads the next rule into the memory 122 from the data store 123. Once the next rule is loaded, the operation 400 repeats blocks 415-445 for the next rule (and each subsequent rule loaded into the memory 122), which includes applying each rule to each corresponding patient health data, as described above with respect to blocks 420-435.

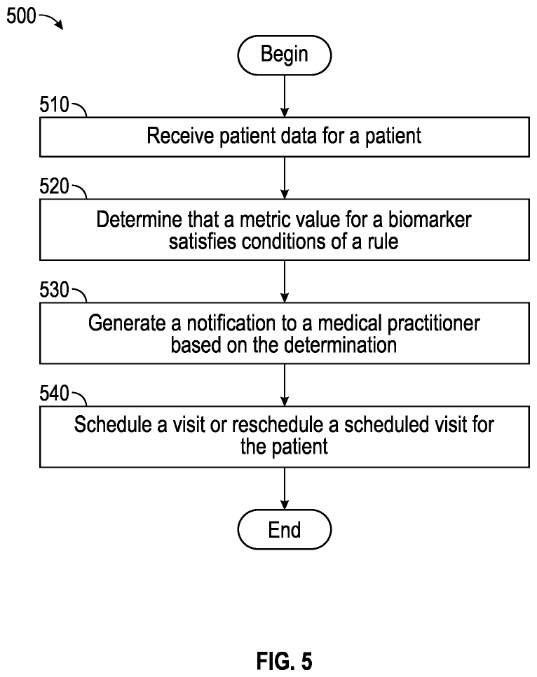

FIG. 5 illustrates a flowchart of operations 500 performed for monitoring a patient's health and notifying a medical practitioner about the patient's health and/or scheduling/rescheduling a visit for the patient based on whether one or more conditions are met, according to certain embodiments described herein. The operations 500 may be performed by the clinic system 120.

At block 510, the clinic system 120 receives patient data for a patient. The patient data may comprise patient health data, such as patient health data 113 (e.g., at least a metric value, such as glucose measurements, for a biomarker, such as blood glucose (e.g., as measured by a monitoring system over a period)). The patient data may further include information about a visit that has been already scheduled for the patient on a future data. The information about the visit may include time and date information and, details of a related patient condition, etc. Further details associated with operations of block 510 were described in relation to block 415 of FIG. 4.

At block 520, the clinic system 120 determines that a metric value for a biomarker satisfies conditions of a rule. As described above, the conditions may be defined by a rule that is created by a medical practitioner for the patient, as described above. In the example of FIG. 3, the conditions correspond to the conditions defined in fields 306A-306C. Further details associated with operations of block 520 were described in relation to blocks 410 of FIG. 4.

At block 530, the clinic system 120 optionally (e.g., automatically) generates a notification and transmits the notification to the medical practitioner, upon determining that the metric value does satisfy the conditions. The notification may comprise an indication that the metric value satisfies the conditions. The notification may also comprise a proposed rescheduled visit. Further details associated with operations of block 530 were described in relation to block 430 of FIG. 4.

At block 540, the clinic system 120 schedules a visit or reschedules a scheduled visit for the patient. In certain embodiments, the clinic system 120 automatically performs the scheduling or rescheduling upon determining that the metric value for the biomarker from the patient data satisfies the conditions. In certain embodiments, the clinic system 120 performs the scheduling or rescheduling upon receiving a confirmation from the medical practitioner in response to the notification that is transmitted to the medical practitioner at block 540. Further details associated with operations of block 510 were described in relation to block 430 of FIG. 4.

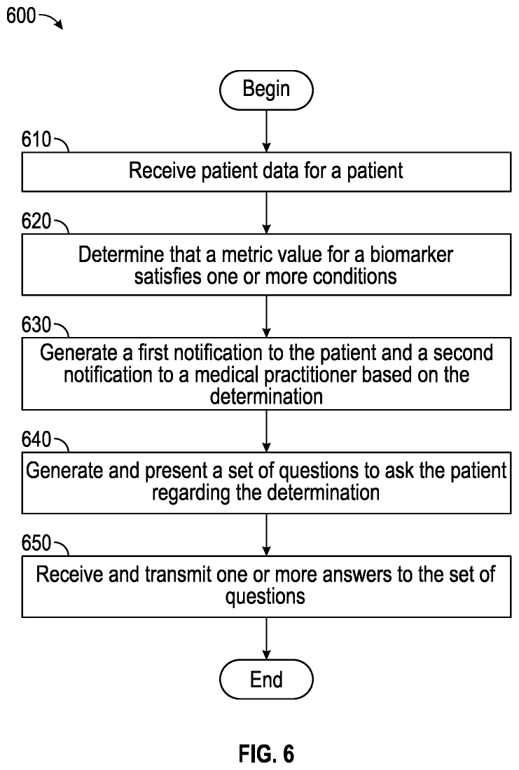

FIG. 6 illustrates a flowchart of operations 600 performed for monitoring a patient's health and proactively alerting the patient and/or a medical practitioner about a change in the patient's health, according to certain embodiments described herein. The operations 600 may be performed by the server system 130.

At block 610, the server system 130 receives patient data for a patient. The patient data may include any information within the patient's profile (e.g., patient profile 110). For example, the server system 130 may receive the patient's health data (e.g., health data 113) on one or more of a continuous, periodic, or on-demand basis. In certain embodiments, the server system 130 may receive patient data in a time period (e.g., a time period prior to the patient's scheduled visit with a medical practitioner, a time period between the patient's last scheduled visit and the patient's next scheduled visit, etc.). The time period may be 7-14 days or some other time period. The patient's health data may include at least one metric value for a biomarker, such as blood glucose measurements (as measured by a monitoring system over a period). For example, a metric indicative of a patient's glucose measurements may include an absolute glucose value, an average glucose value over a time period, a standard deviation of glucose values over time period, a glucose management indicator (GMI) over time period, hemoglobin A1C (HbA1c) over time period, etc. The server system 130 may also receive the patient's scheduling information (e.g., scheduling information 115, including an indication of the patient's next scheduled visit with a medical practitioner).

At block 620, the server system 130 determines that a metric value for a biomarker satisfies one or more conditions. In certain embodiments, at least one of the one or more conditions is based on a metric value having a time in range (TIR) above a threshold. For example, the server system 130 may determine that the patient's absolute glucose values are out of range more than 5% of the time (e.g., TIR is less than 95%). In certain other embodiments, the condition is based on a hyperglycemia metric (e.g., high blood glucose levels) exceeding a certain threshold. In certain other embodiments, the condition is based on a hypoglycemia metric (e.g., low blood glucose levels) exceeding a certain threshold. In certain other embodiments, the condition is based on HbA1c exceeding a threshold. For example, the server system 130 may determine that the patient's HbA1c exceeds a prior HbA1C by greater than a threshold (e.g., 0.4% higher). In another example, the server system 130 may determine that the patient has an increase in GMI that exceeds a threshold (e.g., 0.5%). In certain other embodiments, the condition is based on a comparison of the metric value for the biomarker at different time periods. For example, the server system 130 may determine that a comparison between the patient's absolute glucose values during a first time period and the patient's absolute glucose values during a second time period exceeds a threshold.

At block 630, the server system 130 generates and transmits a first notification to the patient and a second notification to the medical practitioner based on the determination. The first notification may include an indication that a concerning change in the patient's health has been detected. The second notification may also include an indication that a concerning change in the patient's health has been detected, along with an indication of the particular change in the patient's health (e.g., an indication of the metric value that satisfies the condition(s)). An exemplary second notification may indicate that “Patient is experiencing an increase in time spent in a hypoglycemic range,” “Patient is experiencing a decrease in time spent in a hypoglycemic range suggesting improvement in control,” or “Patient has an increase in GMI>0.5% suggesting worsening control.” In certain embodiments, rather than transmit a second notification to the medical practitioner, the server system 130 may generate a report that includes the indication that a concerning change in the patient's health has been detected, along with the indication of the particular change in the patient's health. In such embodiments, the report may be displayed or provided to the medical practitioner (e.g., prior to or during the patient's scheduled visit).

At block 640, the server system 130 generates a set of questions (e.g., question information 116) to ask the patient 102 regarding the determination. In certain embodiments, the server system 130 may present the patient 102 with a set of predefined questions from a standard questionnaire (e.g., Diabetes Treatment Satisfaction questionnaire, CGM Usability questionnaire, SPOTLIGHT questionnaire, etc.). In certain embodiments, the server system 130 may present the patient 102 with a set of questions generated using AI/ML techniques. The set of questions that are selected/generated may be used to obtain information regarding a potential cause for the determination. For example, assuming the patient has an increase in hypoglycemia during a time period, then the server system 130 may query the patient about the patient's food consumption during the time period, activity during the time period, operational status of the patient monitoring system 104 (e.g., whether the patient monitoring system 104 is malfunctioning or operating properly), etc.

At block 650, the server system 130 receives one or more answers to the set of questions and transmits the answers to at least one of (i) a computing system associated with the medical practitioner (e.g., clinic system 120) or (ii) a storage system (e.g., patient data store 140). By providing the medical practitioner with an indication of a change in the patient's health that satisfies certain conditions along with feedback from the user regarding the change in the patient's health, certain embodiments herein enable medical practitioners to make focused decisions and conduct focused discussions with the patient regarding issues the patient may be experiencing during the patient's scheduled visit.

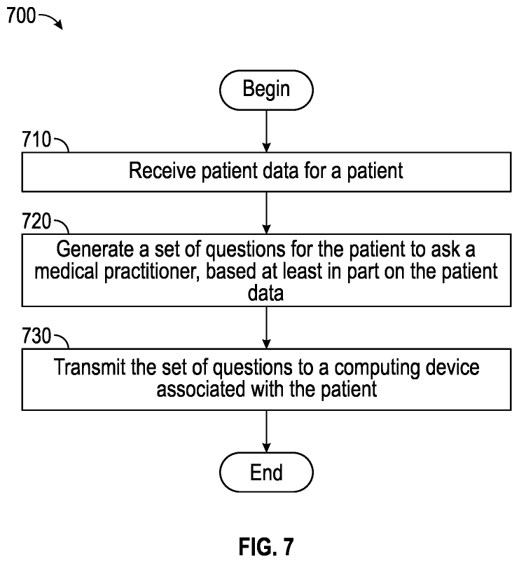

FIG. 7 illustrates a flowchart of operations 700 performed for monitoring a patient's health and proactively prepping a patient for the patient's scheduled visit with a medical practitioner, according to certain embodiments described herein. The operations 700 may be performed by the server system 130.

At block 710, the server system 130 receives patient data for a patient. The patient data may include any information within the patient's profile (e.g., patient profile 110). For example, the server system 130 may receive the patient's health data (e.g., health data 113) on one or more of a continuous, periodic, or on-demand basis. In certain embodiments, the server system 130 may receive patient data in a time period (e.g., 7-14 days or some other time period) prior to the patient's scheduled visit with a medical practitioner.

At block 720, the server system 130 generates a set of questions for the patient to ask a medical practitioner, based at least in part on the patient data. For example, if the patient's health data indicates that the patient has high/low blood glucose values in a time period prior to the patient's scheduled visit, the server system 130 can generate a set of suggested questions regarding high/low blood glucose values for the patient to ask the medical practitioner during the scheduled visit.

At block 730, the server system 130 transmits the set of questions to a computing device associated with the patient (e.g., patient device 107). By providing a set of questions for the patient's scheduled visit in this manner, certain embodiments herein can reduce the likelihood of the patient forgetting which questions to ask during a scheduled visit.

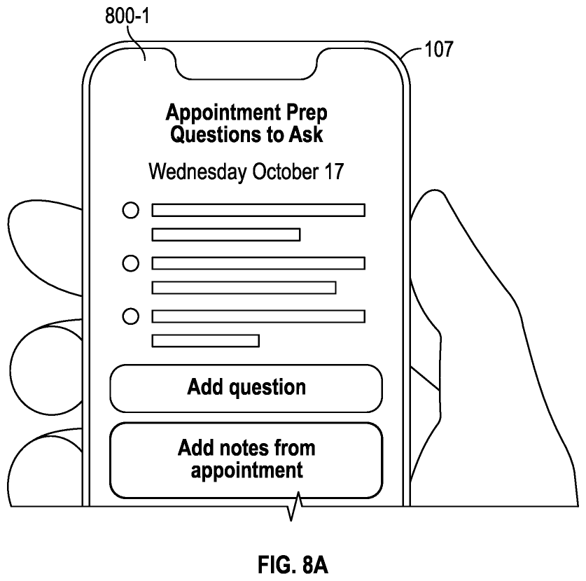

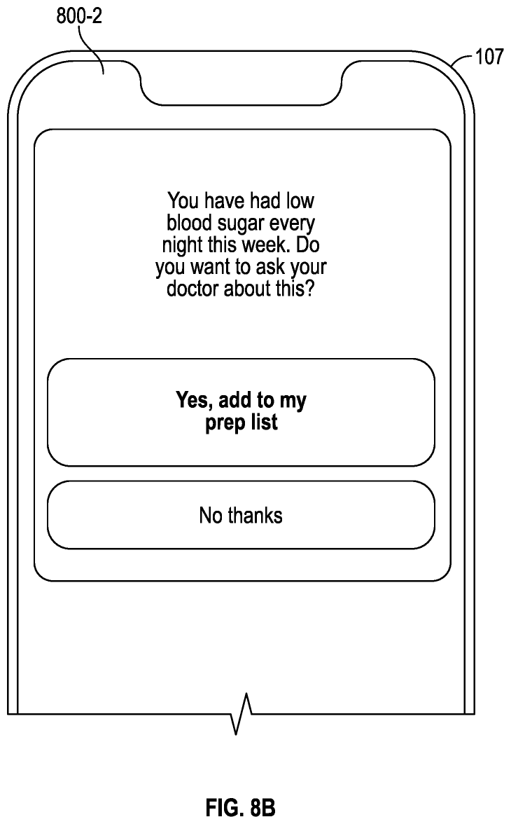

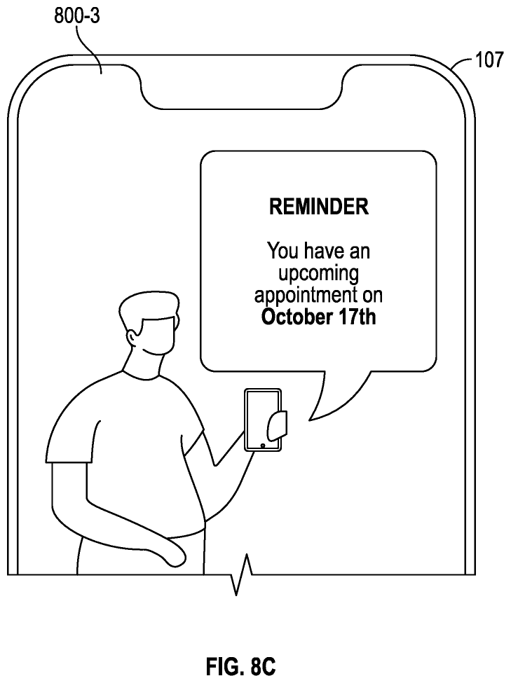

FIGS. 8A-8C illustrate an example scenario for prepping a patient for a scheduled visit with a medical practitioner, according to certain embodiments described herein. In particular, FIGS. 8A-8C depict an example UI 800-1 that may be provided by a clinical support application (e.g., mobile application, web-based application, etc.) provided by a server system 130 for display on a patient device 107. As shown in FIG. 8A, the UI 800-1 may provide a list of questions for the patient 102 to ask the medical practitioner. In certain embodiments, the UI 800-1 depicted in FIG. 8A allows the patient 102 to choose from a list of suggested questions and/or add a patient-defined (or custom) question. Additionally, the UI 800-1 depicted in FIG. 8A allows the patient to add notes to help the patient remember what the medical practitioner advised in between visits.

As shown in FIG. 8B, the UI 800-2 provides talking point suggestions related to trends within the patient's health data during the time period prior to the scheduled visit. Here, in FIG. 8B, for example, the UI 800-2 indicates that the patient has had low blood glucose during the time period prior to the scheduled visit and queries whether the patient would like to discuss their low blood glucose levels during the scheduled visit. The UI 800-2 in FIG. 8B provides a prompt that allows the patient to add the talking point suggestion to a prep list for the scheduled visit. As shown in FIG. 8C, the UI 800-3 may also provide reminders of the patient's scheduled visit with the medical practitioner to help the patient keep track of the scheduled visit.

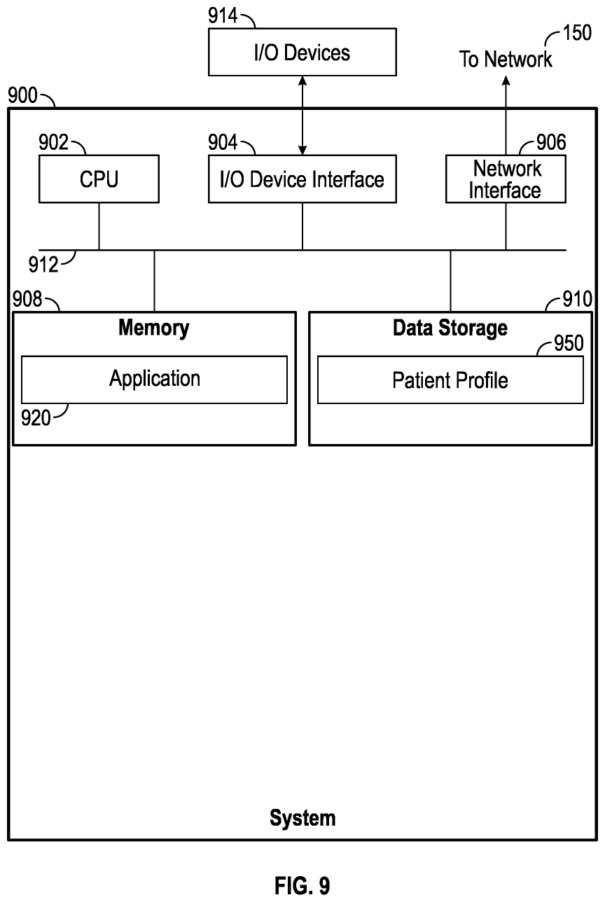

FIG. 9 is a diagram of an embodiment of a processing system that performs or embodies certain aspects described herein. For example, system 900 may correspond to the patient device 107 illustrated in FIG. 1.

As shown, system 900 includes a central processing unit (CPU) 902, one or more I/O device interfaces 904 that may allow for the connection of various I/O devices 914 (e.g., keyboards, displays, mouse devices, pen input, etc.) to the system 900, network interface 906 through which system 900 is connected to the network 150, a memory 908, a data store 910, and an interconnect 912.

The CPU 902 may retrieve and execute programming instructions stored in the memory 908. Similarly, the CPU 902 may retrieve and store application data residing in the memory 908 or accessible via the network 150. The interconnect 912 transmits programming instructions, interactions, and application data, among the CPU 902, I/O device interface 904, network interface 906, and memory 908. The memory 908 is representative of a volatile memory, such as a random access memory, and/or a nonvolatile memory, such as nonvolatile random access memory, phase change random access memory, or the like. As shown, memory 908 includes application 920 (e.g., application 106), which, when executed by the CPU 902 performs the operation described herein with respect to application 106. The data storage 910 may store various data, such as a patient profile 950 stored locally, personalized thresholds, and the like.

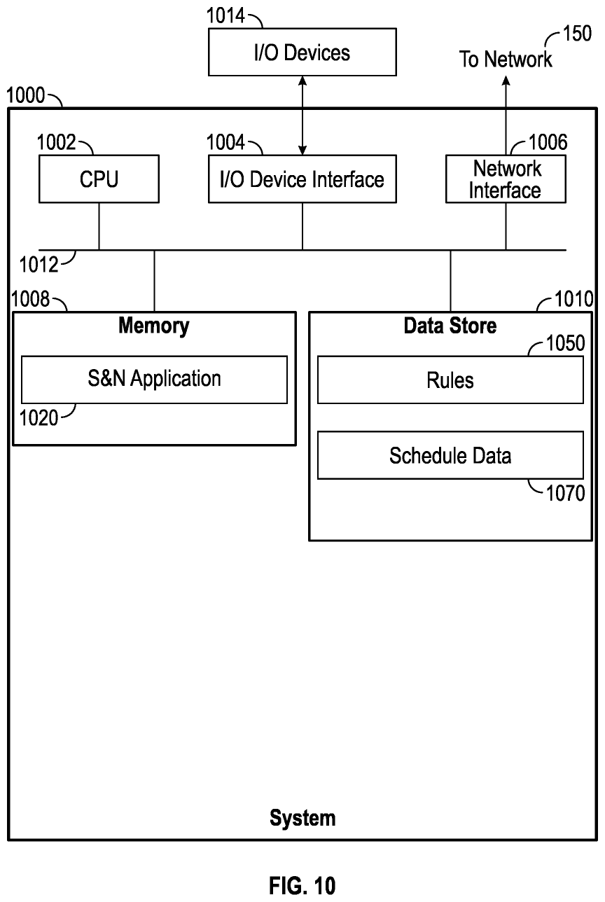

FIG. 10 is a diagram of an embodiment of a processing system that performs or embodies certain aspects described herein. For example, system 1000 may correspond to the clinic system 120 illustrated in FIG. 1.

As shown, system 1000 includes a central processing unit (CPU) 1002, one or more I/O device interfaces 1004 that may allow for the connection of various I/O devices 1014 (e.g., keyboards, displays, mouse devices, pen input, etc.) to the system 1000, network interface 1006 through which system 1000 is connected to the network 150, a memory 1008, a data store 1010, and an interconnect 1012.

The CPU 1002 may retrieve and execute programming instructions stored in the memory 1008. Similarly, the CPU 1002 may retrieve and store application data residing in the memory 1008 or accessible via the network 150. The interconnect 1012 transmits programming instructions, interactions, and application data, among the CPU 1002, I/O device interface 1004, network interface 1006, and memory 1008. The CPU 1002 is included to be representative of a single CPU, multiple CPUs, a single CPU having multiple processing cores, and the like. The CPU 1002 may correspond to the processor 121 of FIG. 1.

The memory 1008 is representative of a volatile memory, such as a random access memory, and/or a nonvolatile memory, such as nonvolatile random access memory, phase change random access memory, or the like. The memory 1008 may correspond to the memory 122. As shown, memory 1008 stores S&N application 1020. The data store 1010 may correspond to the data store 123 and store various data, such as rules 1050 and schedule data 1070.

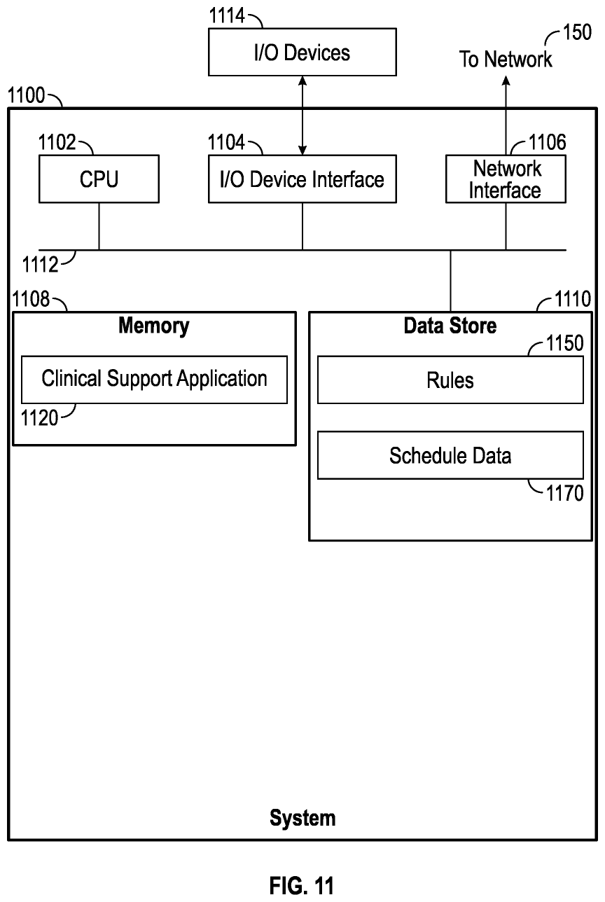

FIG. 11 is a diagram of an embodiment of a processing system that performs or embodies certain aspects described herein. For example, system 1100 may correspond to the server system 130 illustrated in FIG. 1.

As shown, system 1100 includes a central processing unit (CPU) 1102, one or more I/O device interfaces 1104 that may allow for the connection of various I/O devices 1114 (e.g., keyboards, displays, mouse devices, pen input, etc.) to the system 1100, network interface 1106 through which system 1100 is connected to the network 150, a memory 1108, a data store 1110, and an interconnect 1112.

The CPU 1102 may retrieve and execute programming instructions stored in the memory 1108. Similarly, the CPU 1102 may retrieve and store application data residing in the memory 1108 or accessible via the network 150. The interconnect 1112 transmits programming instructions, interactions, and application data, among the CPU 1102, I/O device interface 1104, network interface 1106, and memory 1108. The CPU 1102 is included to be representative of a single CPU, multiple CPUs, a single CPU having multiple processing cores, and the like. The CPU 1102 may correspond to the processor 121 of FIG. 1.

The memory 1108 is representative of a volatile memory, such as a random access memory, and/or a nonvolatile memory, such as nonvolatile random access memory, phase change random access memory, or the like. The memory 1108 may correspond to the memory 132. As shown, memory 1108 stores clinical support application 1120. The data store 1110 may correspond to the data store 133 and store various data, such as rules 1150 and schedule data 1170. The rules 1150 may define a condition(s) under which a notification (or alert) is to be generated and transmitted to the patient 102 and/or the medical practitioner. An exemplary rule 1150 may define that a notification is to be generated and transmitted to the patient 102 and/or the medical practitioner when the patient's blood glucose levels exceeds a threshold (e.g., hyperglycemic condition). Another exemplary rule 1150 may defined that a notification is to be generated and transmitted to the patient 102 and/or the medical practitioner when the patient's blood glucose levels drops below a threshold (e.g., hypoglycemic condition). The schedule data 1170 generally includes information regarding the patient's scheduled visit (e.g., time/date of the patient's scheduled visit).

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer application products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for updating patient scheduling information, the method comprising:
receiving patient data for a patient having a scheduled appointment on a future date, the patient data comprising a metric value for a biomarker and time and date information associated with the scheduled appointment;
comparing the metric value with one or more conditions established based at least in part on a patient history of the patient or population health data; and
after determining that the metric value satisfies at least one of the one or more conditions, rescheduling the scheduled appointment.

2. The method of claim 1, wherein the scheduled appointment comprises one or more of a checkup with a medical practitioner, an out-patient procedure, or an in-patient procedure.

3. The method of claim 1, wherein the biomarker comprises one or more of an analyte, a temperature, a health condition, cholesterol measurement, blood pressure, or heart rate.

4. The method of claim 1, wherein the metric value comprises a value measured by a sensor device or trend data generated by the sensor device over a period.

5. The method of claim 1, further comprising, after determining that the metric value does not satisfy at least one of the one or more conditions, maintaining the scheduled appointment.

6. A method for updating patient scheduling information, the method comprising:
receiving patient data for a patient having a scheduled appointment on a future date, the patient data comprising a metric value for a biomarker as measured by a diagnostic device over a period of time and the scheduled appointment including time and date information;
comparing threshold criteria for the biomarker, the threshold criteria established based at least in part on a patient history of the patient or population health data, to the metric value;
upon determining that the metric value does not satisfy the threshold criteria, automatically generating a first notification comprising a first indication that the metric value does not satisfy the threshold criteria, a first proposal to reschedule the scheduled appointment to one of earlier or later with respect to the future date, and a request for confirmation of the first proposal;
upon determining that the metric value does satisfy the threshold criteria, automatically generating a second notification comprising a second indication that the metric value does satisfy the threshold criteria, a second proposal to reschedule the scheduled appointment to the other of earlier or later with respect to the future date, and a request for confirmation of the second proposal;
upon generating the first notification, communicating the first notification to a medical practitioner; and
upon generating the second notification, communicating the second notification to the medical practitioner.

7. The method of claim 6, wherein the scheduled appointment comprises one or more of a checkup with the medical practitioner, an out-patient procedure, or an in-patient procedure.

8. The method of claim 6, wherein the biomarker comprises one or more of a blood glucose measurement, a measured temperature, a health condition, nutrition details, a medication regimen, cholesterol measurement, blood pressure, or heart rate.

9. The method of claim 6, wherein the metric value comprises a value measured by the diagnostic device or trend data generated based on the metric value over a period.

10. The method of claim 6, wherein the first proposal to reschedule the scheduled appointment comprises one of advancing the scheduled appointment from the future date to a date before the future date or delaying the scheduled appointment from the future date to a date after the future date.

11. The method of claim 6, wherein the second proposal to reschedule the scheduled appointment comprises delaying the scheduled appointment from the future date to a later date or advancing the scheduled appointment from the future date to an earlier date.

12. The method of claim 6, wherein communicating the first notification further comprises communicating the first notification to the patient and communicating the second notification further comprises communicating the second notification to the patient.

13. The method of claim 6, wherein the threshold criteria is further established based on aggregate data from one or more other patient histories with at least one similarity with the patient.

14. A method for updating patient scheduling information, the method comprising:
receiving patient data, measured by a diagnostic device, for a patient having a scheduled appointment on a future date, the patient data comprising a value for a biomarker;
receiving threshold criteria for the biomarker, the threshold criteria established based at least in part on a patient history of the patient and corresponding patient data for at least one other patient;
receiving scheduling information for the patient, the scheduling information including time and date information for the scheduled appointment;
upon receipt of the patient data for the patient having the scheduled appointment, comparing the threshold criteria to a comparison value based on the value for the biomarker relative to the scheduling information for the scheduled appointment and a current date;
upon determining that the comparison value does not satisfy the threshold criteria:
automatically generating a first notification to a healthcare provider, the first notification comprising a first indication that that the biomarker does not satisfy the threshold criteria and a first proposal to reschedule the scheduled appointment for a new date, and
automatically generating a new scheduled appointment for the new date; upon determining that the comparison value does satisfy the threshold criteria:
automatically generating a second alert to the healthcare provider, the second alert comprising a second indication that that the biomarker does satisfy the threshold criteria and a second proposal to reschedule the scheduled appointment for the new date at a later date than the future date, and
automatically generating the new scheduled appointment for the new date;
upon generating the first notification, communicating the first notification to the healthcare provider;
upon generating the second alert, communicating the second alert to the healthcare provider; and
upon receiving confirmation of the first proposal or the second proposal, communicating details regarding the new scheduled appointment to a healthcare provider.

15. The method of claim 14, further comprising conveying a third alert to the patient that the scheduled appointment has been rescheduled due to a determination that the comparison value does satisfy the threshold criteria.

16. The method of claim 14, further comprising conveying a third alert to the patient that the scheduled appointment has been rescheduled due to a determination that the comparison value does not satisfy the threshold criteria.

17. The method of claim 14, further comprising:
generating the comparison value based on:
identifying an expected rate of change for the value over a first period; and
determining the comparison value by applying the expected rate of change for the value to one or more values of a plurality of values for the value in view of the future date.

18. The method of claim 14, wherein the new date is identified based on a prediction of when the biomarker will satisfy the threshold criteria and the current date.

19. The method of claim 14, wherein the biomarker comprises one or more of a blood glucose measurement, a measured temperature, a health condition, nutrition details, a medication regimen, cholesterol measurement, blood pressure, or heart rate.

20. The method of claim 14, wherein the scheduled appointment comprises one or more of a checkup with the medical practitioner, an out-patient procedure, or an in-patient procedure.
